(19) United States
(12) Patent Application Publication
(10) Pub. No.: US 2007/0134232 A1
(43) Pub. Date: Jun. 14, 2007

(54) Topical bite care composition

(76) Inventors: Joel Studin, Great Neck, NY (US); Robert Giuliano, Eastchester, NY (US)

Correspondence Address: FRENKEL & ASSOCIATES, 3975 UNIVERSITY DR., STE. 330, FAIRFAX, VA, 22030, US

(21) Appl. No.: 11/302,878

(22) Filed: Dec. 14, 2005

(51) Int. Cl. A61K 38/46; A61K 31/573; A61K 31/137

(52) U.S. Cl. 424/94.65; 514/171; 514/649

(57) ABSTRACT

The present invention is directed to a topical bite care composition for treating insect bites. The composition contains a steroid and a proteolytic enzyme and/or an antipruritic. The composition can be applied to the skin in any liquid form over the affected area.

### FIELD OF THE INVENTION

The present invention relates to bite care compositions. Particularly, the invention is directed to an improved topical composition for treating insect bites.

### BACKGROUND OF THE INVENTION

Skin is a vital organ that protects animals from external elements. The skin has a “barrier function” that acts to minimize absorption or passage of potentially irritating chemicals through the outer “dead” cell layer into the living skin tissue. The skin can be compromised in a number of ways and can develop many problems. The sensation of itch is one of the most common skin problems experienced by humans and animals. Itch can be defined as a sensation which provokes the desire to scratch the site from which the sensation originates. All skin contains sensory nerves which transmit itch in response to irritating conditions including chemical irritation, environmental exposure (such as that which produces dry, itchy skin) and disease processes such as atopic dermatitis. One common skin itching trigger is an insect bite wherein some toxin or irritant is introduced into the skin triggering the itching sensation.

Topical substances are often used on the skin to reduce the itching sensation or other skin discomforts such as those discomforts caused by insect bites. Compositions for treating or reducing skin irritation are disclosed below. U.S. Pat. No. 4,444,751, discloses a neutralizing composition for sting venoms. The composition comprises a proteolytic enzyme in combination with a carrier suitable for topical application to the skin. Papainase is a preferred proteolytic enzyme used in this composition. U.S. Pat. No. 4,062,937, discloses an insect bite relief preparation comprising an amino acid material in a suitable carrier for topical application to the skin. The amino acid used in the '937 invention is papain. U.S. Pat. No. 5,543,149, discloses a treatment for insect bites comprising compositions provided as solutions, lotions, ointments and salves containing papain or another ingredient.

U.S. Pat. No. 5,728,690, discloses a clear, non-alcoholic hydrocortisone solution. The solution is free of lower alcohols. When applied to skin, the solution will not irritate or dry the skin or give the stinging sensation of an alcohol containing solution. The solution may comprise an antihistamine such as benadryl to alleviate itching. U.S. Pat. No. 6,284,797, discloses a topical treatment for pain and to promote healing of skin and tissue adjacent the skin. The '797 preparation includes capsaicin which is an extract of peppers or chiles and which is a potent local pain killer. Example 9 of '797 shows a sun burn preparation containing among other ingredients capsaicin and hydrocortisone. U.S. Pat. Nos. 5,716,625, 5,804,203 both disclose topical formulations comprising an anti-irritant amount of aqueous-soluble strontium (Sr²⁺) cation, and method for using the same to inhibit skin irritation. The formulation and methods can suppress skin irritation due to chemical or environmental exposure, or due to tissue inflammation, injury or other skin pathology. U.S. Pat. No. 6,139,850 discloses a composition and method for inhibiting skin irritation attributable to chemical irritants or environmental conditions, by the application of an anti-irritant amount of aqueous-soluble strontium cation.

A bite care composition is desired that will treat and/or reduce itching and other adverse affects associated with insect bites.

### SUMMARY OF THE INVENTION

The present invention is directed to a topical composition for treating insect bites. The composition contains a steroid and a proteolytic enzyme and/or an antipruritic. The composition can be applied to the skin in any liquid form over the affected area.

### DETAILED DESCRIPTION

The present invention discloses a bite care composition for treating insect bites. The bite care composition may be used topically, directly over bitten skin to alleviate an itching sensation, relieve pain and/or inflammation due to the bite and provide healing of the affected area. Active ingredients in the composition include a steroid/anti-inflammatory and a proteolytic enzyme and/or an antipruritic.

Steroids are known to have many functions. The steroids having anti-inflammatory effect employed in the present composition are also used to reduce swelling, pain as well as inflammations. Corticosteroids are a natural or synthetic steroid that have anti-inflammatory properties. Synthetic corticosteroids mimic or augment the effects of natural corticosteroid hormones that are produced by adrenal glands. Corticol is a naturally produced example of a corticosteroid. Steroid compounds used in the invention to provide an anti-inflammatory effect may include cortisone, hydrocortisone, fluxinanide, fluoromethalone. Other anti-inflammatory steroid agents include, but are not limited to: triamcinolone and its derivatives (particularly the diacetate, hexacetonide, and acetonide), betamethasone and its derivatives (including particularly the dipropionate, benzoate, sodium phosphate, acetate, and valerate), dexamethasone and its derivatives (particularly the dipropionate and valerate), flunisolide, prednisone and its derivatives (particularly its acetate), prednisolone and its derivatives (particularly its acetate, sodium phosphate and tebutate), methylprednisolone and its derivatives (particularly its acetate and sodium succinate), fluocinolone and its derivatives (particularly the acetonide), diflorasone and its derivatives (particularly the diacetate), halcinonide, desoximetasone (desoxymethasone), diflucortolone and its derivatives (particularly the valerate), flucloronide (fluclorolone acetonide), fluocinonide, fluocortolone, fluprednidene and its derivatives (particularly the acetate), flurandrenolide (flurandrenolone), clobetasol and its derivatives (particularly the propionate), clobetasone and its derivatives (particularly the butyrate), alclometasone, flumethasone and its derivatives (particularly the pivalate), fluocortolone and its derivatives (particularly the hexanoate), amcinonide, beclometasone and its derivatives (particularly the dipropionate), fluticasone and its derivatives (particularly the propionate), difluprednate, and desonide.

The preferred anti-inflammatory steroid used herein is hydrocortisone. As mentioned above, hydrocortisone is a well known chemical that may be produced either by the human adrenal cortex, or synthetically. It is often used in the treatment of a wide array of ailments, including inflammations, allergies and arthritis. Hydrocortisone is used in many topical preparations as a treatment for temporary relief of itching associated with minor skin irritation, inflammation and rashes due to eczema, insect bites, poison ivy, poison oak, poison sumac, soaps, detergents, cosmetics, seborrheic dermatitis, psoriasis and itching in the genital and anal areas of the body. Hydrocortisone speeds up the healing process in wounds or sores that are especially prone to swelling, such as in the case of an insect bite or sting. Hydrocortisone is also helpful in applications where the sores are not particularly prone to swelling. The steroid may be present in the topical composition in a range about 0.1-5.0 wt. %, preferably in a range about 0.1-1.0 wt. %, and more preferably in a range about 0.3-0.6 wt. %.

The composition of this invention includes a proteolytic enzyme. The proteolytic enzyme used in the present composition, for example, may be selected from the group consisting of papain, chymopapain, hyaluronidase, desoxyribonuclease, trypsin, chymotrypsin, bromelain and mixtures thereof. Other hormone enzymes can be used if effective for application in topical compositions. The preferred proteolytic enzyme used herein is papain. Papain is a cysteine protease isolated from papaya fruit. The cysteine proteases are a class of proteolytic enzymes that use nucleophilic catalysis in hydrolyzing a peptide bond. Papain is known to be effective in neutralizing toxicants introduced into the body by stings, bites or the like by either puncturing or cutting the skin.

Proteolytic enzymes are useful in digesting proteins such as toxins. It is the presence of these proteins that trigger the itching sensation when introduced into the body. The digestion of these toxins into smaller particles allow for the lessening of the itching sensation in the body. The proteolytic enzyme, such as papain for example, may be present in the composition in a range about 0.01-5.0 wt. %, preferably in a range about 0.05-1.0 wt. %, and more preferably in a range about 0.1-0.5 wt. %.

At least one antipruritic may be added to the composition. The preferred antipruritic of the present invention employs a strontium metal cation. The strontium metal cation is useful in reducing the incidence and severity of skin irritations. It is believed that the strontium cation may reduce irritation by interacting with epidermal nerve cells to prevent or counteract the sensation of irritation, and/or by interfering with irritation-inducing components of skin cells that are triggered by the skin irritant. Thus, the cation may alter the ability of epidermal nerve cells to depolarize or repolarize, as for example by blocking or interfering with ion channel or pump operation or by altering the transmembranal action potential, or the cation may interfere with the transmission of nerve impulses from one nerve cell to another (as by suppressing neurotransmitter release).

Preferred embodiments of the present invention utilize an anti-irritant amount of the strontium cation accompanied (as in the form of a salt) by one or more ionizing anionic species, preferably an acidic anion species such as a chloride, nitrate, acetate, gluconate or oxalate anion, dissolved or dispersed in an appropriate vehicle. The anti-irritant effects of the cations of the invention can be optimized by suitable selection of accompanying anionic species. Especially preferred cation-anion pairs include strontium chloride, strontium nitrate, and strontium acetate, with strontium nitrate being most preferred. The antipruritic strontium nitrate may be present in the composition in a range about 0.1-5.0 wt. %, preferably in a range about 0.2-1.0 wt. %, and more preferably in a range about 0.2-0.75 wt. %.

The venom or toxins introduced into the skin cause histamines to be released, which ultimately cause an itch. Accordingly, additional antipruritic may be included in the composition such as antihistaminic agents. Antihistaminic agents, i.e., drugs capable of antagonizing the in vivo effects of histamine have been known for decades. The classical antihistamines act by competitively antagonizing the effects of histamine at H1-receptor sites. While antihistamines have traditionally been administered orally and in some instances parentally, topical antihistaminic preparations, particularly incorporating diphenhydramine, which is sold commercially as Benadryl®, have been used to reduce pruritus caused by, e.g., fixed drug eruptions, contact dermatitis and insect bites. Such topical antihistaminic preparations have also been utilized to treat the erythema and edema of insect bites, poison ivy and so on.

Other substances may be added to composition of the present invention including organic solvents suitable for topical administration. Solvents used herein dissolve the ingredients of the composition and act as a vehicle for the ingredients. Suitable solvents may be selected from the lower alcohols (ethanol, isopropanol), glycols such as propylene, ethylene and polyethylene glycols (liquids at room temperature). Further solvents include hydrocarbons, aldehydes, ketones, etc. Preferred organic solvents used herein include ethyl alcohol, propylene glycol, and acetone. The range of organic solvents range from 0.1 to 50 wt. %. Water can also be used or added.

Penetration enhancers, specifically hydrophilic penetration enhancers may also be added to the composition. Hydrophilic penetration enhancers can be, for example, an alcohol, a nonionic solubilizer or an emulsifier. Suitable hydrophilic components include, but are not limited to, ethylene glycol, propylene glycol, dimethyl sulfoxide (DMSO), dimethyl polysiloxane (DMPX), oleic acid, caprylic acid, isopropyl alcohol, 1-octanol, ethanol (denatured or anhydrous), and other pharamceutical grade or absolute alcohols with the exception of methanol. Additional skin penetration enhancers include decyl methyl sulfoxide, N-dodecyl pyrrolidone, decanol, dodecanol, an organic acid such as oleic acid, or the like. Dimethyl Sulfoxide is the preferred penetration enhancer. Penetration enhancers are employed in the composition in a range of 0.5 to 5 wt. %.

Anesthetic agents may also be used in the topical bite care composition of the present invention. Anesthetics are effective in preventing or relieving pain by interrupting nerve conduction. These anesthetic agents may be those known in the art including but not limited to benzocaine, bupivacaine, chloroprocaine, cinchocaine, cocaine, dexivacaine, diamocaine, dibucaine, etidocaine, hexylcaine, levobupivacaine, lidocaine, pontocaine, mepivacaine, oxethazaine, prilocalne, procaine, proparacaine, propoxycaine, pyrrocaine, risocaine, rodocaine, ropivacaine, and tetracaine; and pharmaceutically acceptable derivatives thereof, and combinations thereof.

Derivatives of these compounds, such as pharmaceutically acceptable salts and esters are also of particular interest, for example, bupivacaine HCl, chloroprocaine HCl, diamocaine cyclamate, dibucaine HCl, dyclonine HCl, etidocaine HCl, levobupivacaine HCl, lidocaine HCl, mepivacaine HCl, pramoxine HCl, prilocalne HCl, procaine HCl, proparacaine HCl, propoxycaine HCl, ropivacaine HCl, tetracaine HCl, and so forth, and so forth. The preferred anesthetic agent used herein is Lidocaine HCl. The anesthetics may be employed in the composition of the present invention in a range of about 0.05% to 5.0%, preferably from about 0.1% to 2.0%, more preferably from about 0.2% to 1.0%, by weight.

These anesthetic agents are suitable for topical, transdermal delivery (e.g., can be delivered through body surfaces and membranes, including skin) and induces the desired local or systemic effect include, by way of example and not limitation, treatment for burns, contact dermatitis, insect bites, pain, pruritus, skin rash, wounds, and so forth.

Other substances may be added to composition of the present invention including buffering agents, preservatives, thickening agents, colorants, etc.

The present invention may have any form including liquid such as a solution or emulsion, paste, gel or cream. The composition can be applied by swab, brush, roller, spray, dropper, etc.

### EXAMPLE

The following table includes an example of a composition of the present invention, which is presented for illustrative purposes only.

TABLE 1

| Ingredient | Wt. % |
|---|---|
| Diphenhydramine (Benadryl ®) | 2.0 |
| Lidocaine HCl | 0.5 |
| Hydrocortisone | 0.5 |
| Strontium Nitrate | 0.5 |
| Papain | 0.2 |
| Ethyl Alcohol | 33.0 |
| Propylene Glycol | 30.0 |
| Acetone | 3.0 |
| Dimethyl Sulfoxide | 2.0 |
| Water qs | 100.0 |

1. A bite care composition comprising
(a) about 0.1-5.0 wt % of a steroid; and
(b) about 0.01-5.0 wt % of a proteolytic enzyme.

2. (canceled)

3. The composition of claim 1, wherein the steroid is present in the composition in a range of about 0.1-1.0 wt. %.

4. The composition of claim 1, wherein the steroid is hydrocortisone.

5. (canceled)

6. The composition of claim 1, wherein the proteolytic enzyme is papain.

7. The composition of claim 1, further comprising an anesthetic.

8. The composition of claim 1, further comprising an organic solvent.

9. The composition of claim 8, wherein the organic solvent is an alcohol, glycol, or ketone, or mixtures thereof.

10. The composition of claim 1, further comprising a penetration enhancer.

11. The composition of claim 1, further comprising at least one antipruritic.

12. The composition of claim 11, wherein the antipruritic comprises strontium cation, antihistamine or mixtures thereof.

13.-20. (canceled)

21. A method of treating insect bites comprising applying onto skin that has been affected by an insect bite, a composition comprising a steroid and a proteolytic enzyme.

22. The method of claim 21, wherein said composition comprises about 0.1-5.0 wt % of a steroid; about 0.01-5.0 wt % of a proteolytic enzyme.

23. The method of claim 21, wherein the steroid is hydrocortisone.

24. The method of claim 21, wherein the proteolytic enzyme is papain.

25. The method of claim 21, wherein said composition further comprises an anesthetic.

26. The method of claim 21, wherein said composition further comprises an organic solvent.

27. The method of claim 21, wherein said composition further comprises at least one antipruritic selected from strontium cation, antihistamine or mixtures thereof.
